(19) United States
(12) Patent Application Publication
(10) Pub. No.: US 2009/0325917 A1
(43) Pub. Date: Dec. 31, 2009

(54) Pharmaceutical Compositions and Nasal Spray Incorporating Anhydrous Mometasone Furoate

(75) Inventors: Amar Lulla, Mumbai (IN); Geena Malhotra, Mumbai (IN)

Correspondence Address: CONLEY ROSE, P.C., 5601 GRANITE PARKWAY, SUITE 750, PLANO, TX, 75024, US

(73) Assignee: CIPLA LIMITED, Mumbai (IN)

(21) Appl. No.: 12/446,156

(22) PCT Filed: Oct. 19, 2007
(86) PCT No.: PCT/GB2007/004022

§ 371 Date: Aug. 6, 2009

(30) Foreign Application Priority Data
Oct. 19, 2006 (IN) 1742/MUM/2006

(51) Int. Cl. A61K 31/58

(52) U.S. Cl. 514/172

(57) ABSTRACT

A stable aqueous pharmaceutical composition comprising anhydrous mometasone furoate and a pharmaceutically acceptable carrier.

The present invention relates to a pharmaceutical composition useful for preventing or minimizing allergic reactions. More particularly, the invention relates to a stable pharmaceutical composition comprising anhydrous mometasone furoate, which may be administered in the form of a nasal spray. The invention also relates to a process for the preparation of such a composition and to a method of treatment of a subject in need thereof.

Many people suffer from seasonal and perennial allergic rhinitis worldwide. Symptoms of seasonal and perennial allergic rhinitis include nasal itch, congestion, runny nose, sneezing and watery eyes. Seasonal allergic rhinitis is commonly known as “hay fever”. It is caused by allergens which are present in the air at specific times of the year. Perennial allergic rhinitis is caused by allergens which are present in the environment year-round. Examples of such allergens are dust mites, mold, mildew, and pet dander.

Such forms of rhinitis are treated with medicaments such as, for example, steroidal anti-inflammatory agents. Mometasone furoate is an example of a widely used steroidal anti-inflammatory agent. Such an agent is generally used by spraying it into the nasal passages of the human patient where it deposits on surfaces of the mucosa which line the nasal cavities. In this position, the medicament exerts its pharmacological action as it is in contact with bodily tissues and interacts with steroid receptors.

For maximum effectiveness, the nature of the pharmaceutical composition containing the medicament should be such that the medicament is delivered readily to all portions of the nasal cavities (the target tissues) where it performs its pharmacological function. In addition, the medicament should remain in contact with the target tissues for relatively long periods of time. The longer the medicament remains in contact with the target tissues, the greater the opportunity for the medicament to perform its function. In order to remain in contact with the target tissues, the medicament must be capable of resisting those forces in the nasal passages that function to remove particles from the nose. Such forces, referred to as “mucocillary clearance”, are recognized as being extremely effective in removing particles from the nose in a rapid manner, for example, within 10-30 minutes from the time the particles enter the nose.

It is particularly important that such pharmaceutical compositions have satisfactory stability and shelf-life properties, such that they remain stable and active for as long as possible.

Other desired characteristics of the pharmaceutical composition are that it should not contain ingredients which cause the user discomfort, and that it not include constituents that are considered to be detrimental to the environment, for example, ozone depletors.

US 2005/0186144 describes methods for treating rhinosinusitis of the upper airway passages in patients afflicted with said disease, which comprises administering at least once a day to the surfaces of said passages of said patients an amount of aerosolized particles of mometasone furoate as a monotherapy for treating said disease.

WO 2004/020289 describes methods of introducing a non-aqueous suspension or solution of a medicament, such as mometasone furoate anhydrous into a metered dose inhaler for administration to the lungs. The medicament is introduced as an alcoholic solution, typically together with a surfactant.

U.S. Pat. No. 6,127,353 describes an aqueous composition comprising a stable crystalline form of mometasone, namely mometasone furoate monohydrate. The inventors of U.S. Pat. No. 6,127,353 found that a composition containing anhydrous mometasone furoate in aqueous solution was unstable, and converted to a different crystalline form after storage at 35° C.

It is an object of the present invention to provide stable compositions containing anhydrous mometasone furoate. In particular It is an object of the present invention to provide a mometasone furoate composition that does not change its crystalline form. It is also an object of the invention to provide an aqueous composition of anhydrous mometasone furoate which can be administered to the nasal mucosa.

We have surprisingly found that aqueous mometasone furoate anhydrous compositions can be formed which are stable and maintain the same crystalline form in solution for long periods of time; These compositions can be formed as nasal sprays.

In a first aspect, the present invention provides an aqueous pharmaceutical composition comprising anhydrous mometasone furoate in a pharmaceutically acceptable carrier.

The pharmaceutical acceptable carrier preferably comprises water.

The composition is preferably in the form of a suspension. The suspension is preferably an aqueous suspension.

The composition is preferably in the form of a nasal spray.

In accordance with the present invention, it is possible to make aqueous pharmaceutical compositions which are stable. The anhydrous form of anhydrous mometasone furoate in the pharmaceutical compositions according to the invention does not change its crystallinity during storage, and has a long shelf-life.

The pharmaceutical composition of the present invention may comprise from 0.1 to 10.0 mg of anhydrous mometasone furoate per gram of suspension.

The compositions according to the invention are aqueous, which means that the vehicle used to suspend the mometasone is water. Preferably the vehicle is substantially entirely water, i.e, the composition is substantially free of any organic carrier, such as an organic solvent.

The composition according to the invention preferably comprises at least 95.35 wt % water, more preferably at least 95.36 wt % water, more preferably at least 95.368 wt % water.

The anhydrous mometasone furoate can be manufactured by known methods, such as those described in U.S. Pat. No. 4,472,393.

The pharmaceutically acceptable carrier of the present Invention may further comprise, inter alia, suitable excipients and auxiliaries, such as preservatives, suspending agents, viscosifiers, isotonicity agents, buffering agents, humectants, etc.

The pharmaceutical composition may further comprise one or more preservative. It is preferred that the preservative comprises one or more substance selected from the group consisting of benzalkonium chloride, benzethonium chloride, methyl p-hydroxybenzoate, ethyl p-hydroxybenzoate, butyl p-hydroxybenzoate, propyl p-hydroxybenzoate, thimerosal, sodium dehydroacetate and myristyl-gamma-picolinium chloride, sodium benzoate, potassium benzoate, potassium sorbate. Preferably the preservative is benzalkonium chloride.

The pharmaceutical composition may further comprise one or more buffering agents. The buffering agent may comprise one or more substance selected from the group consisting of sodium hydrogenphosphate, potassium dihydrogenphosphate, dipotassium phosphate, anhydrous sodium dihydrogenphosphate, crystalline sodium dihydrogenphosphate, boric acid, borax, sodium acetate, citric acid, citric anhydride, sodium citrate, sodium glutamate and creatinine. Preferably the buffering agent is citric acid and sodium citrate. The citric acid may be anhydrous.

The pharmaceutical composition may further comprise one or more humectants. The humectants may be selected from one or more substance selected from the group consisting of glycerol, propylene glycol, sorbitol, carboxyvinyl polymer, polyethylene glycol.

The composition may further comprise one or more suspending agents. The suspending agents may be selected from one or more of sodium carboxymethyl cellulose, xanthan gum, microcrystalline cellulose, carragenan, veegum, tragacanth, bentonite, methylcellulose, and polyethylene glycols. A preferred suspending agent is a mixture of microcrystalline cellulose and carboxymethylcellulose.

The pharmaceutical composition may further comprise one or more wetting agents. Since mometasone furoate is hydrophobic it is preferable to include a pharmaceutically acceptable dispersing agent which functions to wet the particles of medicament to facilitate dispersion thereof in the aqueous phase of the composition. The present invention may comprise suitable dispersing agents selected from the group consisting of one or more of fatty alcohols, esters, and ethers, including, for example, those sold under the trademarks Pluronic, Tergitol, Span, and Tween. It is preferred to use a hydrophilic, non-ionic surfactant, like Polysorbate 80.

For the purpose of nasal administration a mildly acidic pH is generally preferred. Preferably the compositions of the present invention have a pH in the range of 3 to 6, more preferably in the range of 3.5 to 5.

The compositions of the present invention also possess appropriate isotonicity and viscosity. Preferably compositions according to the present invention have an osmotic pressure of 270 to 350 mOsm/liter. Any suitable isotonic agent and/or thickening agent may be used to achieve appropriate isotonicity and/or viscosity.

In an embodiment, the composition comprises from 0.01 and 0.10 wt % anhydrous mometasone furoate, based on the weight of the composition. Preferably the composition comprises from 0.1 to 10 wt % anhydrous mometasone furoate. In a preferred embodiment, the composition comprises 0.05 wt % anhydrous mometasone furoate.

In an embodiment, the composition comprises 0.001 to 0.05 wt % of a preservative. In a preferred embodiment, the composition comprises 0.01 wt % preservative.

In an embodiment, the composition comprises 0.01 to 1.0 wt % of a buffering agent. In a preferred embodiment, the composition comprises approximately 0.475 wt % buffering agent. The buffering agent may be a mixture of buffering agents. In a particularly preferred embodiment, the buffering agent comprise 0.195 wt % citric acid and 0.277 wt % sodium citrate.

In an embodiment, the composition comprises 0.05 to 5 wt % of a humectant. Preferably the composition comprises 0.1 to 5 wt % humectant. More preferably, the composition comprises approximately 2.0 wt % humectant.

In an embodiment, the composition comprises 0.1 to 4 wt % of a suspending agent. Preferably the composition comprises 1.0 to 5 wt % of a suspending agent. More preferably the composition comprises 1.0 to 3 wt % of a suspending agent. More preferably still, the composition comprises 2 wt % of a suspending agent.

In an embodiment, the composition comprises 0.001 to 0.2 wt % of a wetting/dispersing agent. Preferably, the composition comprises 0.01 wt % of a wetting/dispersing agent.

The remainder of the composition may comprise water.

The composition according to the invention may be alcohol-free. In particular, the composition may be free from ethanol, ethyl alcohol, phenylethyl alcohol and the like.

The use of a surfactant in the formulation can give rise to undesirable problems with foaming. We have found that it is possible to solve this problem by formulating the composition such that it is substantially free of surfactant.

Thus according to another aspect of the invention there is provided an aqueous pharmaceutical composition comprising anhydrous mometasone furoate and a pharmaceutically acceptable carrier, wherein said composition is substantially free of a surfactant.

Preferably there is less than 0.05 wt % surfactant in the composition, more preferably less than 0:04 wt %, more preferably less than or equal to 0.02 wt %, more preferably less than or equal to 0.015 wt % surfactant, and still more preferably less than or equal to 0.011 wt % surfactant. In a preferred embodiment there is less than or equal to 0.01 wt % surfactant. In a another embodiment, there may be less than or equal to 0.005 wt % surfactant. In an embodiment, there may be no detectable amount of surfactant in the composition.

In a preferred embodiment of the present invention benzalkonium chloride is used as a preservative. We have unexpectedly found, however, that the surfactant properties of benzalkonium chloride (or another preservative having surfactant properties) alone are sufficient to provide an adequate surfactant effect Thus, the composition can be formulated without any additional surfactant, thereby avoiding the foaming problems associated with an additional surfactant.

Thus, according to another aspect of the invention there is provided an aqueous pharmaceutical composition comprising anhydrous mometasone furoate and a pharmaceutically acceptable carrier, wherein said composition contains at least one preservative which has surfactant properties, and wherein the composition is substantially free of any additional surfactant other than the or each preservative.

It is preferred that the preservative is benzalkonium chloride. It is further preferred that the amount of preservative in the composition is less than 0.05 wt %, more preferably less than 0.04 wt %, more preferably less than or equal to 0.02 wt %, more preferably less than or equal to 0.015 wt %, and still more preferably less than or equal to 0.011 wt %. In a preferred embodiment there is less than or equal to 0.01 wt % preservative. In another embodiment, there may be less than or equal to 0.005 wt % preservative.

In certain embodiments, there may be a small amount of additional surfactant (which is not a preservative). In such embodiments, the amount of additional surfactant is preferably less than 0.02 wt %, or less than or equal to 0.01 wt %,.or less than or equal to 0.005 wt %. It is preferred that if a detectable amount of an additional surfactant (which is not a preservative) is present, then the total amount of the preservative and the additional surfactant is less than 0.05 wt %, more preferably less than 0.04 wt % and most preferably less than or equal to 0.02 wt %.

In an embodiment, there may be no detectable amount of any other surfactant in the composition. These compositions free of surfactant (other than preservative) preferably include the other excipients as described above.

All the compositions according to the invention provide formulations in which the mometasone furoate is suspended therein.

For the purpose of nasal application a composition according to the present invention is preferably included in a suitable container. The container is preferably provided with means enabling the application of the contained composition to the nasal mucosa. Suitable applicators are known in the art and include those aiding the administration of liquid nasal compositions in a solution or spray form. Since the dosing should be done as accurately as possible, spray form is a more suitable medium. Spray form administrators suitable for use include atomizers, pump-atomizers, aerosols and the like.

It will be appreciated, therefore, that the present invention further provides a nasal spray dispenser comprising (i) a housing containing a composition comprising mometasone furoate anhydrous in a pharmaceutically acceptable liquid carrier; and (ii) means enabling the application of the composition from within the housing to the nasal mucosa.

The stability of the compositions in accordance with the present invention may be defined by standard methods. The anhydrous crystalline form is stable at room temperature. In particular, when subjected to temperatures of 25° for a period of three months and at 40° C. for a period of three months, the formulation was stable, in that the crystalline form of the anhydrous mometasone furoate in the composition described herein does not change.

In another method, when rotated for five days at 35° C. and an additional four weeks at room temperature (typically approximately 20 to 25° C., more typically 22 to 25° C., and most typically 25° C.) the crystalline form of the anhydrous mometasone furoate in the composition described herein does not change.

The crystalline form may be assessed using X-ray diffraction methods.

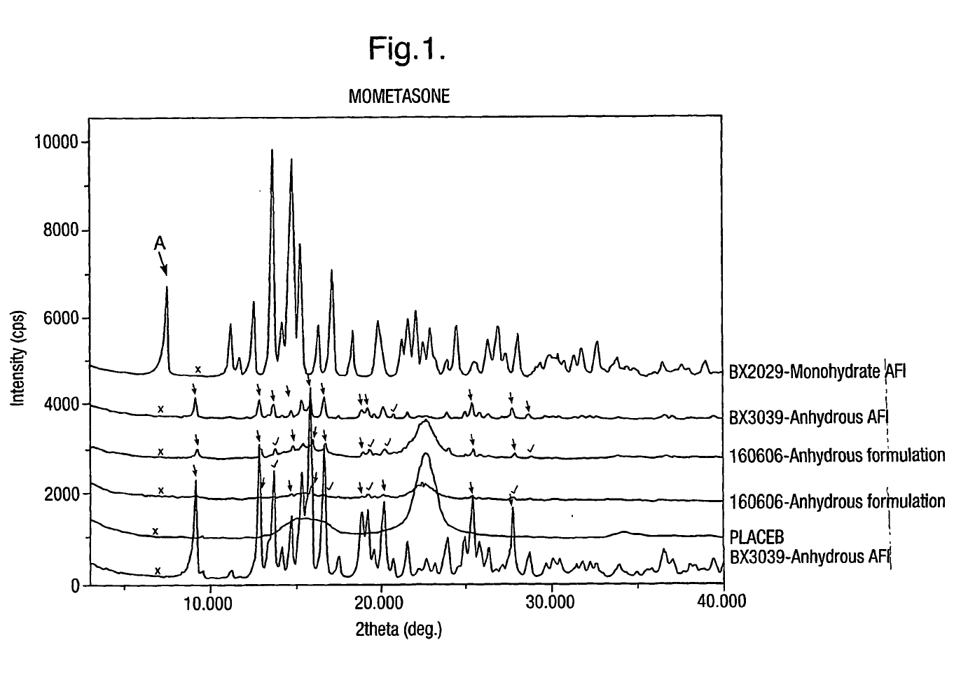

FIG. 1 shows XRD spectra for mometasone furoate monohydrate (BX 2029), mometasone furoate anhydrous API (BX 3039), and mometasone furoate anhydrous formulation (160606). The monohydrate peak is clearly visible in the BX 2029 trace, and is absent in the remaining pattern, indicating the presence of anhydrous form in the formulation. Stability of the mometasone furoate anhydrous API was confirmed by boiling in water (with and without the surfactant Tween 80) for two hours whilst stirring at 70 deg in a water bath, followed by 2 hours at 70 deg on a magnetic stirrer. The solution was cooled to room temperature, filtered and the residue containing the API was dried at 25° C. under vacuum. The dried sample was tested by X-ray diffraction, and the XRD pattern was found to be concordant with that of mometasone furoate anhydrous.

The present invention also provides, a process for preparing a pharmaceutical composition substantially as hereinbefore described, which process comprises combining anhydrous mometasone furoate with a pharmaceutically acceptable carrier.

The present invention also provides a method of administering mometasone furoate anhydrous to a subject requiring mometasone treatment, which method comprises administering via the nasal route to said subject a pharmaceutical composition as described herein. In particular, the treatment is of allergic rhinitis, and optionally disorders associated with allergic rhinitis.

The present invention also provides, for use in the manufacture of a medicament for the treatment of a disease state requiring mometasone treatment, especially allergic rhinitis, mometasone furoate anhydrous in a pharmaceutically acceptable liquid carrier.

Stability of the mometasone furoate anhydrous API was confirmed by boiling in water (with and without the surfactant Tween 80) for two hours whilst stirring at 70 deg in a water bath, followed by 2 hours at 70 deg on a magnetic stirrer. The solution was cooled to room temperature, filtered and the residue containing the API was dried at 25° C. under vacuum. The dried sample was tested by X-ray diffraction, and the XRD pattern was found to be concordant with that of mometasone furoate anhydrous.

### EXAMPLE 1

| Sr. | | |
|---|---|---|
| No. | Ingredients | Qty. (% w/w) |
| 1. | Mometasone Furoate anhydrous | 0.050 |
| 2. | Benzalkonium Chloride | 0.01% w/w |
| | As Benzalkonium chloride solution 10% w/v | 0.1% v/w |
| 3. | Anhydrous citric acid | 0.195 |
| 4. | Glycerol | 2.0 |
| 5. | Dispersible cellulose | 2.0 |
| 6. | Polysorbate 80 | 0.01 |
| 7. | Sodium Citrate | 0.277 |
| 8. | Water for injection | qs |

1. Dispersible cellulose was dissolved in water to obtain a lump-free suspension.
2. To this was added glycerol under stirring.
3. A separate solution of citric acid was made and added to the main bulk.
4. This was followed by the addition of a separate solution of sodium citrate to the main bulk.
5. Polysorbate was dissolved in water, to this the mometasone furoate anhydrous was added to get a uniform slurry.
6. Benzalkonium chloride (as 10%w/v solution) was added to the above slurry.
7. This drug slurry was added to the main bulk of cellulose dispersion under continuous stirring.
8. The pH was adjusted and the volume was made up.

### EXAMPLE 2

Mometasone Furoate Nasal Spray 0.05% w/w (50 mcg/Spray)

| | Quantity |
|---|---|
| COMPONENT | (% w/w) |
| Mometasone furoate anhydrous | 0.05 |
| Benzalkonium chloride NF | 0.02 |
| Citric acid monohydrate USP | 0.2 |
| Glycerin USP | 2.1 |
| Microcrystalline cellulose and Carboxymethylcellulose | 2.0 |
| sodium NF | |
| Sodium citrate dihydrate USP | 0.28 |
| Water for injection | q.s. to 100 gms. |

1. The ingredients i.e. microcrystalline cellulose and sodium carboxymethyl cellulose were sieved and sifted and dispersed in part quantity pre-cooled water for injection and stirred.
2. Glycerine was added to the main bulk, mixed and stirred.
3. Similarly, citric acid monohydrate, sodium citrate and benzalkonium chloride solution were added, individually, in part quantity of pre-cooled water for injection, and stirred and mixed with the bulk solution
4. A slurry of mometasone furoate anhydrous was prepared, stirred and mixed with the bulk solution.
5. Subsequently, checked and recorded the pH of the bulk solution and made the volume with water for injection and mixed.

It will be appreciated that the invention can be modified within the spirit and scope of the claims.

1. An aqueous pharmaceutical composition comprising anhydrous mometasone furoate and a pharmaceutically acceptable carrier.

2. The pharmaceutical composition according to claim 1, wherein the anhydrous mometasone furoate does not change in its crystalline form when subjected to stability testing.

3. The pharmaceutical composition according to claim 1, wherein the composition is stable, which means that when subjected to stability testing formation of a crystalline material which is different from the anhydrous mometasone furoate is not observed in suspension.

4. The pharmaceutical composition according to claim 1, wherein the mometasone furoate is suspended in a vehicle which is substantially water, and which is substantially free of any organic solvent.

5.-40. (canceled)

41. The pharmaceutical composition according to claim 1, which is in the form of a nasal spray.

42. The pharmaceutical composition according to claim 1, wherein the pharmaceutically acceptable carrier further comprises a preservative.

43. The pharmaceutical composition according to claim 42, wherein the preservative comprises one or more of phenol, benzyl alcohol, phenylethyl alcohol, chlorhexidine, benzalkonium chloride, benzethonium chloride, methyl p-hydroxybenzoate, ethyl p-hydroxybenzoate, butyl p-hydroxybenzoate, propyl p-hydroxybenzoate, ethanol, chlorobutanol, thimerosal, sodium dehydroacetate and myristyl-gamma-picolinium chloride, sodium benzoate, potassium benzoate, potassium sorbate.

44. The pharmaceutical composition according to claim 42, wherein the preservative is benzalkonium chloride.

45. The pharmaceutical composition according to claim 1, wherein the pharmaceutically acceptable carrier comprises at least one of a suspending agent, a wetting or dispersing agent, a viscosifier, an isotonicity agent, a buffering agent, and a humectant.

46. The pharmaceutical composition according to claim 1, comprising:
(a) from 0.01 to 0.10 wt % of anhydrous mometasone furoate;
(b) from 0.1 to 4 wt % of suspending agent;
(c) from 0.05 to 5 wt % of humectant;
(d) from 0.01 to 1.0 wt % of buffering agent;
(e) from 0.001 to 0.05 wt % of preservative;
(f) from 0.001 to 0.2 wt % of wetting or dispersing agent; and
optionally (g) up to 0.05 wt % of surfactant.

47. The pharmaceutical composition according to claim 1, wherein the composition has a pH in the range of 3 to 6.

48. The pharmaceutical composition according to claim 1, wherein the composition has a pH in the range of 3.5 to 5.

49. The pharmaceutical composition according to claim 1, wherein the composition is contained within a suitable container for application by spraying to the nasal mucosa.

50. The pharmaceutical composition according to claim 1, which contains no alcohol.

51. An aqueous pharmaceutical composition comprising anhydrous mometasone furoate and a pharmaceutically acceptable carrier, wherein said composition is substantially free of a surfactant.

52. An aqueous pharmaceutical composition comprising anhydrous mometasone furoate and a pharmaceutically acceptable carrier, wherein said composition contains at least one preservative which has surfactant properties, and wherein the composition is substantially free of any additional surfactant other than the or each preservative.

53. The aqueous pharmaceutical composition according to claim 52, wherein the preservative is present in an amount of 0.02 wt % or less, based on the weight of the composition, preferably 0.01 wt % or less, more preferably 0.005 wt % or less.

54. A pharmaceutical composition comprising:
(a) 0.05% wt of anhydrous mometasone furoate;
(b) 0.02% wt of benzalkonium chloride;
(c) 0.2% wt of citric acid monohydrate;
(d) 2.1% wt of glycerin;
(e) 2.0% wt of microcrystalline cellulose and carboxymethyl cellulose sodium mixture; and
(f) 0.28% sodium citrate dihydrate;
wherein said composition is substantially free of a surfactant except benzalkonium chloride.

55. The nasal spray dispenser comprising (i) a housing containing a pharmaceutical composition according to claim 1; and (ii) means enabling the application of the composition from within the housing to the nasal mucosa.
